(19) United States
(12) Patent Application Publication
(10) Pub. No.: US 2021/0156942 A1
(43) Pub. Date: May 27, 2021

(54) DISTINGUISHING BETWEEN EXTRAVASCULAR AND INTRAVASCULAR CONTRAST POOLS

(71) Applicant: Oregon Health & Science University, Portland, OR (US)

(72) Inventors: Ramon Francisco Barajas, JR., Happy Valley, OR (US); Daniel Schwartz, Portland, OR (US)

(21) Appl. No.: 16/953,895

(22) Filed: Nov. 20, 2020

Related U.S. Application Data
(60) Provisional application No. 62/938,789, filed on Nov. 21, 2019.

(51) Int. Cl. G01R 33/56; G06T 7/00; G16H 30/40; G16H 50/20; A61B 5/00; A61B 5/055; G01R 33/50

(52) U.S. Cl. CPC G01R 33/5601; G06T 7/0016; G16H 30/40; G16H 50/20; A61B 5/0042; A61B 2576/026; G01R 33/50; G06T 2207/30104; G06T 2207/30096; G06T 2207/10088; G06T 2207/20224; A61B 5/055

(57) ABSTRACT

Embodiments of the present disclosure relate to methods, apparatus, and computer readable medium for detecting an immune factor responsive to a contrast agent. In some embodiments, pre-contrast image data, early timepoint image data, and delayed timepoint image data are received. In some embodiments, an early timepoint map is generated using a comparison of the pre-contrast image data to the early timepoint image data, and a delayed timepoint map is generated using a comparison of the pre-contrast image data to the delayed timepoint image data, where the early timepoint map represents intravascular contrast, the delayed timepoint map represents the intravascular contrast and extravascular contrast, and the extravascular contrast indicates the immune factor. In some embodiments, a combined immune factor map is generated by voxel-wise subtraction of the early timepoint map from the delayed timepoint map.

### RELATED APPLICATION

This application claims priority benefit of U.S. Provisional Patent Application No. 62/938,789, filed Nov. 21, 2019, which is hereby incorporated by reference in its entirety.

### FEDERALLY SPONSORED RESEARCH OR DEVELOPMENT

This invention was made with government support under R01 CA137488 awarded by the National Institutes of Health. The government has certain rights in the invention.

### TECHNICAL FIELD

This disclosure relates generally to analysis of magnetic resonance imaging (MRI) images and, more particularly, to detection of an immune factor.

### BACKGROUND INFORMATION

Despite advances in surgical resection and Stupp protocol adjuvant chemoradiotherapy (e.g., CRT; temozolomide with conformal irradiation), the median overall survival is currently about 14.2 months in isocitrate dehydrogenase (IDH) wild-type glioblastoma. Gadolinium-based contrast (GBCA) enhanced magnetic resonance imaging (MRI) is a technique employed for therapeutic monitoring of glioblastoma. Following CRT, some patients undergo an inflammatory response that manifests as progressive GBCA enhancement, commonly termed pseudoprogression. The radiographic appearance of this inflammatory process is a result of neurovascular unit disruption and subsequent leakage of GBCA into the extravascular tissue space. The relationship between glioblastoma pseudoprogression and overall survival is not yet completely known; some investigators have reported an association between improved overall survival and pseudoprogression, whereas others have not observed this survival benefit. This discrepancy is likely a factor of the retrospective nature of pseudoprogression diagnosis and the lack of standardized histological criteria by which this disease process is defined. Also, pseudoprogression cannot be distinguished from true glioblastoma regrowth using GBCA-MRI. As such, definitive diagnosis has entailed either surgical biopsy or watchful waiting for diminishing GBCA enhancement, both of which can effectively delay definitive therapy for true disease recurrence and potentially adversely impact overall survival.

Tumor-associated macrophages (TAMs) are a constituent of the immune response to glioblastoma therapy, accounting for up to 40% of the cellular mass. TAMs are iron recyclers having inflammatory properties directly linked to phagocytosis. Ultra-small superparamagnetic iron oxide (USPIO) contrast enhanced MRI is suited for the detection of neuroinflammation due to the TAMs phagocytic capabilities. TAM-mediated neuroinflammation has been localized by so-called delayed timepoint MRI twenty-four hours after intravenous USPIO ferumoxytol (Feraheme®, available from AMAG Pharmaceuticals, Inc. of Waltham, Mass.) administration in preclinical glioblastoma models.

Ferumoxytol is FDA-approved for intravenous iron supplementation and has off label uses as an MRI contrast agent to evaluate central nervous system (CNS) disease. Ferumoxytol-induced MRI signal change contributes information relating to concentration and localization (intra- and extravascular) of the agent, and the timing of imaging with respect to the administration can provide additional information.

So-called early timepoint MRI (immediately following intravenous ferumoxytol administration) allows for the quantification and localization of intravascular agent; however, the prolonged circulating half-life of approximately 15 hours may limit facile interpretation of signal changes in the glioblastoma microenvironment in delayed timepoint of imaging up to 48 hours later. More specifically, in a paper titled “Quantification of Macrophages in High-Grade Gliomas by Using Ferumoxytol-enhanced MRI: A Pilot Study,” Iv et al. have reported that glioblastoma regions devoid of macrophage accumulation demonstrate T2*-weighted signal changes in the delayed timepoint of ferumoxytol imaging. (Radiology. Published online: Nov. 6, 2018; in print: January 2019; vol. 290, No. 1, pp. 198-206.)

### SUMMARY OF THE DISCLOSURE

The ability to differentiate intravascular from extravascular contrast signal within tissues allows for a more accurate assessment of tissue microvasculature and tissue cellular components. When using a contrast agent (e.g., nanoparticles, such as iron nanoparticles, for example), the disclosed techniques allow for the specific localization of the contrast agent within an extravascular compartment of a region of interest, where it is preferentially taken up by innate immune cells. Accordingly, disclosed are techniques that, among other things, address a current lack of a clinically applicable biomarker for immune activity. Immune activity can be, for example, glioblastoma immune activity. The disclosed imaging metric specific to inflammatory changes improves glioblastoma clinical outcomes. Disclosed techniques are also applicable to analyzing immune activity for diseases other than glioblastoma.

Embodiments of this disclosure, for example, specifically localize and differentiate extravascular from intravascular MRI contrast within tissues using noninvasive T2* weighting and susceptibility weighted sequences. In other words, disclosed embodiments allow for the specific localization of innate immune cells within tissue without signal contamination from intravascular contrast.

Some disclosed embodiments are useful for quantifying and monitoring innate immune cell infiltration within tissues.

Some embodiments of this disclosure provide the ability to locate immune cells in applications throughout the body in many disease processes, including disease assessment and treatment.

For example, embodiments of the present disclosure relate to methods, apparatus, and computer readable medium for detecting an immune factor responsive to a contrast agent. In some embodiments, pre-contrast image data, early timepoint image data, and delayed timepoint image data is received, the pre-contrast image data representing image data at an initial time related to intravenous administration of the contrast agent, the early timepoint image data representing image data at an early timepoint following intravenous administration of the contrast agent, and the delayed timepoint image data representing image data at delayed timepoint following intravenous administration of the contrast agent. In some embodiments, an early timepoint map is generated using a comparison of the pre-contrast image data to the early timepoint image data, and a delayed timepoint map is generated using a comparison of the pre-contrast image data to the delayed timepoint image data, the early timepoint map representing intravascular contrast, the delayed timepoint map representing the intravascular contrast and extravascular contrast, and the extravascular contrast indicating the immune factor. In some embodiments, a combined immune factor map is generated by voxel-wise subtraction of the early timepoint map from the delayed timepoint map, the combined immune factor map suppressing the representation of the intravascular contrast of the delayed timepoint map and localizing the representation of the extravascular contrast of the delayed timepoint map indicating the immune factor.

In some embodiments, the receiving comprises receiving, via a network, one or more of the pre-contrast image data, early timepoint image data, and delayed timepoint image data.

In some embodiments, the receiving comprises receiving, via direct communication, one or more of the pre-contrast image data, early timepoint image data, and delayed timepoint image data.

Additional aspects and advantages will be apparent from the following detailed description of embodiments, which proceeds with reference to the accompanying drawings.

### BRIEF DESCRIPTION OF THE DRAWINGS

FIG. 1 is an annotated pictorial example of a Segregation and Extravascular Localization of Ferumoxytol Imaging (SELFI) processing pipeline.

FIG. 2 is a hybrid set of pictorial and corresponding histogram graphs diagram showing specific extravascular ferumoxytol accumulation localized.

FIGS. 3A and 3B are first and second portions of a table showing cohort demographics and treatment regimen.

FIGS. 4A and 4B are graphs showing how techniques of the present disclosure discriminate extravascular from intravascular ferumoxytol contrast pools.

FIG. 5 is a block diagram of a computing device according to embodiments of the present disclosure.

### DETAILED DESCRIPTION OF EMBODIMENTS

It is the inventors' present belief that observed signal changes (e.g., for both T1 and T2* images) in the delayed timepoint represent a combination of two spatially separate and functionally distinct contrast (e.g., ferumoxytol) pools: (1) residual intravascular contrast, and (2) extravascular accumulation of contrast within TAMs. Therefore, uncorrected delayed timepoint contrast enhanced-MRI may lead to falsely positive inflammatory hotspots due to persistent intravascular agent. Accordingly, disclosed are techniques that use an imaging and analytic approach that, in some embodiments, facilitate isolation of an extravascular signal from a residual intravascular signal present in the delayed timepoint by employing an image subtraction technique. Intravascular contrast is, for example, contrast that is situated within blood vessels in tissue. Extravascular contrast is, for example, contrast that is situated outside blood vessels in tissue.

In some embodiments, the aforementioned techniques are deployed in connection with T2* weighted imaging, which can be, for example, susceptibility weighted imaging (SWI). SWI is a three-dimension (3D) high spatial resolution gradient echo MRI sequence sensitive to T2* relaxation and, due to its short repetition time, is also marginally sensitive to T1 effects. The disclosed embodiments include employing multiple time point measurements of ferumoxytol-enhanced SWI signal to produce high resolution maps of putatively isolated extravascular ferumoxytol contrast. This technique is referred to as segregation and extravascular localization of ferumoxytol imaging (SELFI).

It should be appreciated that, in light of this disclosure, other types are contrast agents are also suitable for detecting macrophages or other types of immune factors. For example, gold nanoparticles and other types of contrast are suitable. For conciseness, however, the following passages describe embodiments employing ferumoxytol.

Also described are results of an experiment testing the hypothesis that SELFI distinguishes extravascular ferumoxytol contrast signal from residual intravascular signal at the 24-hour delayed imaging timepoint. Using a well characterized cohort of glioblastoma patients previously treated with Stupp protocol CRT who received concurrent GBCA- and ferumoxytol-enhanced MRI for the clinical suspicion of glioblastoma disease progression, results show that (1) signal on delayed timepoint SWI is partly a reflection of remaining intravascular contrast, (2) the generation of SELFI maps allows for the localization of accumulated extravascular ferumoxytol signal that is observed in the delayed timepoint of imaging, and (3) Positive SELFI (SELFI+) values, in part, tend to stratify overall survival in patients with glioblastoma.

### Image Acquisition

By way of example, the aforementioned image analysis of embodiments of the present disclosure is described in connection with patient experiments in which they underwent 3.0T MRI examinations (MRI equipment is available from Philips Healthcare North America as the Ingenia 3.0T MR system) using one of two imaging protocols.

A first protocol (12 patients) entailed MRI performed over three consecutive days.

A second protocol (11 patients) entailed MRI performed on two consecutive days.

The following imaging parameters were used for SWI that was performed prior to, immediately following (early timepoint), and about 24 hours (delayed timepoint) after intravenous ferumoxytol administration (over 20 min infusion at a dose of up to 510 mg): TR/TE/FA=26 ms/20 ms/15°, FOV=(210 mm)², imaging matrix=(512)², in plane resolution=(0.41)², 116 contiguous axial slices with 2.2 mm slice thickness, 1.1 mm overlap. The superscripts here refer to the two spatial dimensions (e.g., x and y coordinate axes) in which the data was obtained.

The following parameters were used for gradient-echo 3D T1-weighted images acquired pre- and post-gadoteridol gadolinium chelate (ProHance®, available from Bracco Diagnostic Inc. of Princeton, N.J.; 0.1 mmol/kg) and ferumoxytol administration: TR/TE/FA=8.14 ms/3.72 ms/8°, 100 contiguous 2 mm thick axial slices. GBCA-enhanced sequences were performed before ferumoxytol administration. MRI measurements were completed on the same instrument for each participant.

### Generation of SELFI Maps

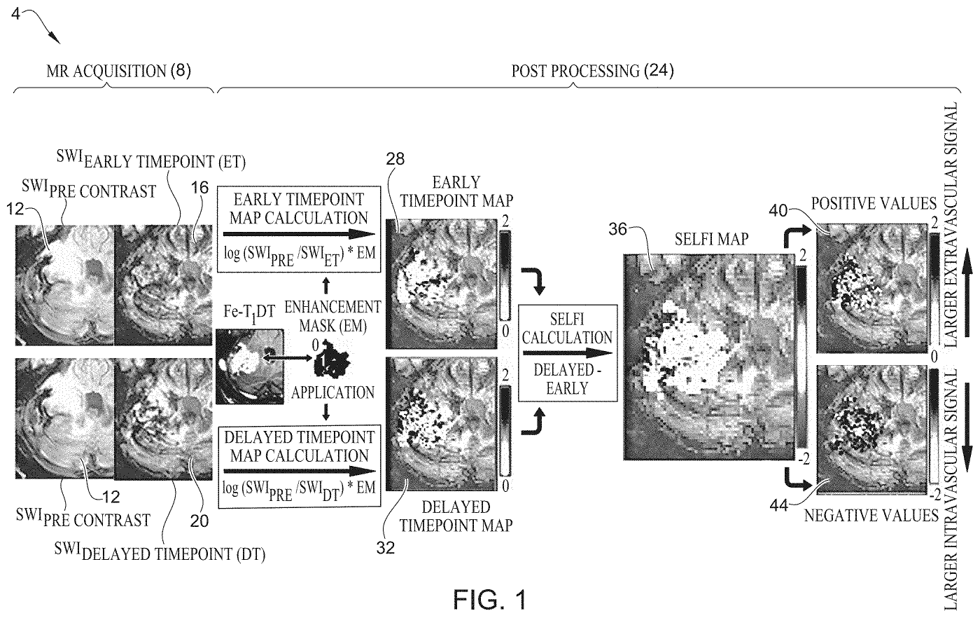

FIG. 1 shows an annotated pictorial example 4 of a SELFI processing pipeline. An MR acquisition 8 of susceptibility weighted imaging is performed prior to, immediately after, and 24 hours following ferumoxytol administration, in the present example. These imaging times are also referred to as pre-contrast, an early timepoint (ET, also referred to as a primarily intravascular timepoint), and a delayed timepoint (DT, also referred to as mixed intra- and extra-vascular timepoint).

In some embodiments, the pre-contrast timepoint corresponds to a stage at an initial time related to intravenous administration of a contrast agent (i.e., before an operative effect of such agent). For example, in some embodiments, the initial time can be prior to (e.g., before any) intravenous administration of a contrast agent or shortly after (e.g., about zero seconds to about 15 minutes after) the intravenous administration of a contrast agent. (Note that the term “about” includes the value stated.) ET corresponds to an early timepoint about 15 minutes to about 20 minutes after the intravenous administration of the contrast (or alternatively within about 20 minutes after the intravenous administration of the contrast), and the DT corresponds to a delayed timepoint about 24 hours to about 72 hours after the intravenous administration of the contrast. Thus, the present example shows a pre-contrast image 12, an early timepoint image 16, and a delayed timepoint image 20 to be processed.

In some embodiments, SWI magnitude images (e.g., pre-contrast 12, early timepoint 16, and delayed timepoint 20) are aligned to a weighted image. (using the 3dAllineate program, for example). The 3dAllineate software is available from the National Institutes of Health (NIH) as a component of the AFINI suite of software tools. In some embodiments, the weighted image is a non-contrast enhanced T1-weighted volume image. In some embodiments the weighted image is a T2* weighted image. In some embodiments, the weighted image is a T2* weighted post gadolinium enhanced volume image. In general, registration entails a two-step process. First, a coarse resolution search is performed to approximate a three-dimensional spatial transform that results in the to-be-registered bodies to be close to one another. Second, a fine resolution search in a six-parameter space after center of mass matching is performed to match the to-be-registered bodies as closely as possible. A cross correlation cost function can be used at each iteration of the two-step process to evaluate current parameter sets. A final dataset is transformed and interpolated using a sinc function with a Hanning function that has a seven-voxel window, for example.

Post processing 24 is performed. Here, early timepoint map 28 and delayed timepoint map 32 are calculated on a voxel-wise basis as the log of the quotient of the post to pre-contrast images. As used herein, “log” should be understood as a natural log, which is used so that nonlinearities in ratio variables are linearized such that the ratios are equidistant and the dependent variable is not weighted in favor of the denominator. For example, the ratios of 5/2 and 2/5 are not equidistant from one until the log is performed (e.g., log₁₀(5/2)=0.40 and log₁₀(2/5)=−0.40). This allows for centering of the displacement of the relationship evenly around zero irrespective of which variable is in the numerator or denominator. Likewise, the introduction of a log transform equilibrates the magnitude of an effect (both positive and negative) between two hypothetical voxels.” Early and delayed timepoint maps 28 and 32 are created by taking the natural log of the ratio of a pre-contrast voxel (acquired from pre-contrast SWI magnitude image) to the same post-contrast voxel (acquired from either early timepoint image 16 or delayed timepoint image 20), such that the magnitude of a voxel's value is proportional to the concentration of contrast.

In some embodiments, a negative value that results from each natural log calculation is attributable to voxels having an SWI signal showing an increase after the administration of contrast agent. This is optionally considered noise and removed from both early and delayed timepoint maps. In experimentation, less than five percent of the voxels were removed.

In some embodiments, resulting values are multiplied by an enhancement mask (EM) factor (e.g., a zero or a one) corresponding to a region of interest in one or both of the early timepoint map 28 and delayed timepoint map 32. For instance, the EM factor is derived from the contrast enhanced T1-weighted image and provides a non-specific search space within which the SELFI maps are calculated. For example, a neurological radiologist having 10 years of experience reviewed contrast enhanced T1-weighted volumes (GBCA and delayed ferumoxytol) and manually marked a region of interest (ROI) covering possible enhancement in both T1-weighted data sets. These preliminary ROIs were then automatically segmented into contrast-enhancing and non-enhancing voxels. Results of the segmentation were visually inspected, and no manual intervention was provided.

Post processing 24 also includes the generation of SELFI maps. A first SELFI map 36 is created by taking the voxel-wise difference of delayed timepoint map 32 from early timepoint map 28 (using the 3dcalc program, for example, which is another component of AFINI). Accordingly, first SELFI map 36 is represented by the following equation:

$$SELFI = log ( \frac{{SWI}_{pre - contrast timepoint}}{{SWI}_{delayed timepoint}} ) - log ( \frac{{SWI}_{pre - contrast timepoint}}{{SWI}_{early timepoint}} )$$

where SWI_{pre-contrast timepoint} is a voxel from pre-contrast SWI image 12, SWI_{dearly timepoint} is a voxel from early timepoint SWI image 16, and SWI_{delayed timepoint} is a voxel from delayed timepoint SWI image 20 (captured about 24 hours after contrast administration).

The process that has been highlighted is an a priori censorship of voxels that display an unexpected pattern of enhancement (for example, SWI signal is increased after contrast administration).

As explained, first SELFI map 36 is calculated as the difference of delayed timepoint map 32 from early timepoint map 28. SELFI map 36 can be further segmented into a map 40 of positive SELFI values (SELFI+ values) (e.g., in which the delayed timepoint signal is greater than early timepoint signal, and thereby represents an extravascular or so-called SELFI+ map) and map 44 of negative SELFI values (SELFI− values) (e.g., in which the early timepoint signal is greater than the delayed timepoint signal, and thereby represents an intravascular or so-called SELFI− map).

To generate aggregate SELFI+ and SELFI− values from maps 40 and 44, mean values from each map are calculated within both the GBCA and ferumoxytol contrast-enhancing region of interest (ROI) (e.g., using the 3dmaskave program, which is also available in the AFNI software). A voxel-wise mathematical calculation, for example, entails summing all voxels within the mask, and the sum is divided by the number of voxels within the mask. The mathematical definition of this process is provided in equations in the next paragraph and is illustrated as SELFI+ (reflecting an aggregate value from positive SELFI value map 40) and SELFI− (reflecting an aggregate value from negative SELFI value map 44).

The negative (“SELFI−”) value map 44 and positive (“SELFI+”) value map 40 of the first SELFI map 36 are calculated separately such that the absolute value of each positive and negative voxel in map 36 is proportional to the relative contribution of that voxel:

$$SELFI += \frac{1}{n} {\sum }_{{SELFI}_{i} > 0}^{n} i$$

$$SELFI -= \frac{1}{n} {\sum }_{{SELFI}_{i} < 0}^{n} i$$

where n is the number of voxels within map 36 defined by the enhancement mask and i is a particular voxel within map 36 defined by the enhancement mask.

To provide a single aggregate “per tumor” score of both the magnitude and spatial extent of inflammatory infiltrate, primarily for the purpose of testing against overall survival (see later paragraphs discussing survival analysis under “Example Experimentation” subsection), SELFI+ values of SELFI value map 40 were scaled by the proportion of voxels that were positive relative to the extent of ferumoxytol enhanced T1. This scaling pegs SELFI+ values to a normalizable quantity. The extent of T1-weighted enhancement can be used as a proxy for a so-called severity of disease metric or assessment, which has prognostic value. In other words, the extent of enhancement on a contrast-enhanced T1-weighted volume image is indicative of the spatial extent of the disease, and the extent of enhancement can be used to further refine the severity of disease metric or assessment to equilibrate disease situations (e.g., equilibrate a situation where high inflammation is present in a few voxels with a situation where medium inflammation is present in many voxels).

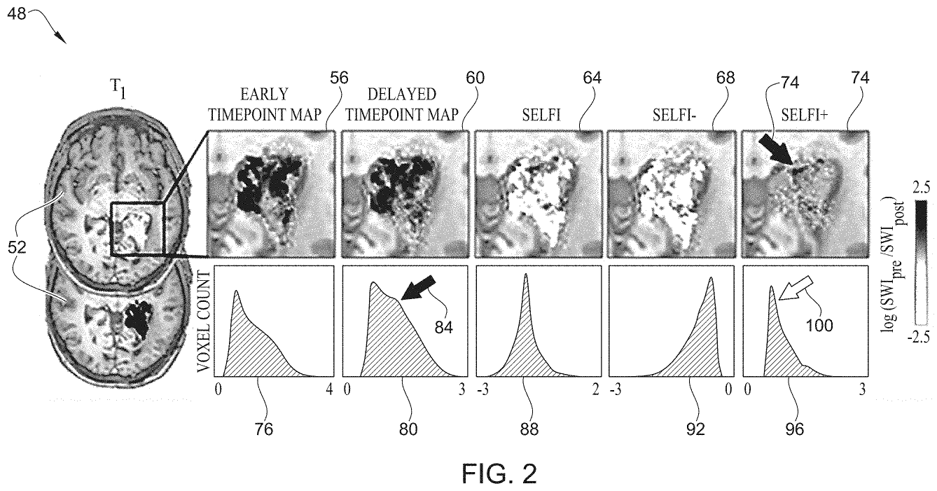

FIG. 2 shows an example 48 where an enhanced image 52 is analyzed to generate an early timepoint map 56, a delayed timepoint map 60, SELFI maps (first SELFI map 64, SELFI− value map 68, and SELFI+ value map 72) and corresponding voxel-wise histograms 76, 80, 88, 92, and 96 from a single patient. The Y axis of histograms 76, 80, 88, 92, and 96 represent the respective voxel count of maps 56, 60, 64, 68, and 72. The X axis of histograms 76, 80, 88, 92, and 96 represent SELFI values generated based on equations set forth above. In FIG. 2, SWIₚₒₛₜ can be either SWI_{early timepoint} from an early timepoint SWI image associated with enhanced image 52 or SWI_{delayed timepoint} from a delayed timepoint SWI image associated with enhanced image 52.

In FIG. 2, specific extravascular ferumoxytol accumulation is localized by SELFI+ values in map 72. Image 52 can be a T1-weighted ferumoxytol enhanced image and automated enhancement segmentation mask, for example. Similarities are observed between early timepoint map 56 and delayed timepoint map 60. The hump shown in histogram 80 of delayed timepoint map 60 (shown by closed arrow 84) represents remaining intravascular ferumoxytol; this signal is removed in the SELFI+ map (rightmost histogram 96, shown by open arrow 100). The specific extravascular signal that arises from ferumoxytol contrast accumulation in the delayed timepoint is only clearly visualized by the SELFI technique in the SELFI+ value map 72 (shown by red closed arrow 74 in map 72).

### Example Experimentation

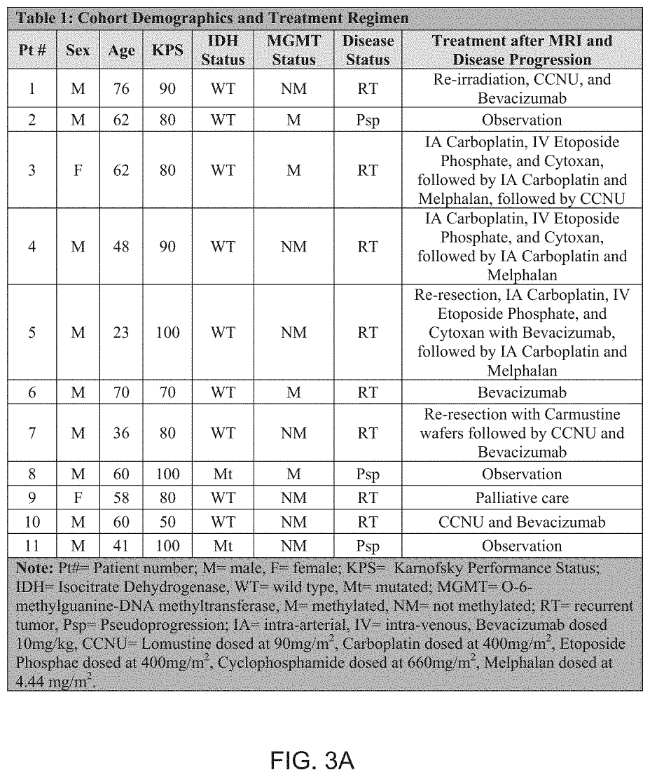

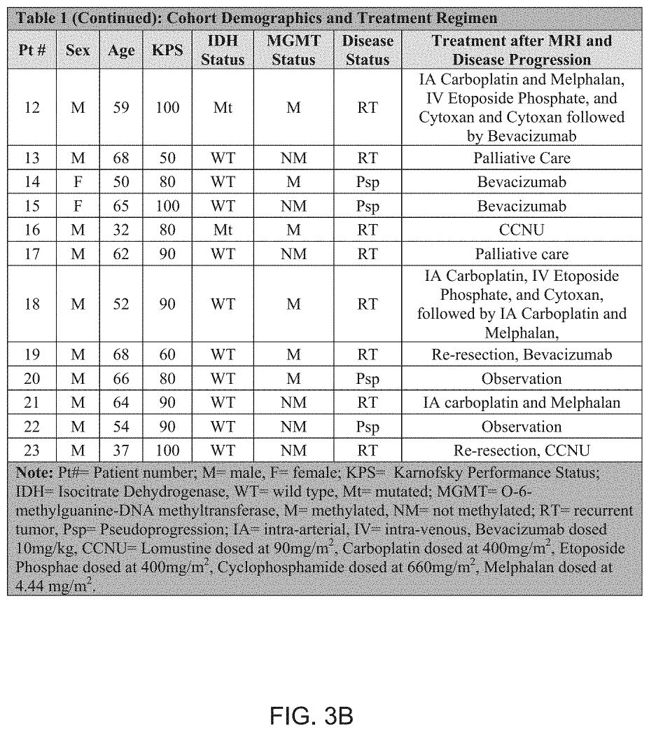

As shown in table 1 of FIGS. 3A and 3B, twenty-three patients with glioblastoma progression following Stupp protocol chemoradiotherapy were tested in an experiment. All patients underwent ferumoxytol-enhanced susceptibility weighted imaging (SWI) magnetic resonance imaging, in addition to standard T1-weighted sequences. Imaging was performed prior to, immediately after (early timepoint), and 24-hours after (delayed timepoint) intravenous ferumoxytol administration. SELFI maps were generated as the voxel-wise difference of the 24-hour delayed timepoint map from the early timepoint map. Prespecified end points included negative and positive portions of SELFI maps. Pearson's r- and Student's t-tests were performed. In terms of survival analysis, overall survival was classified in IDH wild type glioblastoma from the date of surgical diagnosis until death or last follow-up date for patients without an event. Survival analysis was performed by using multivariate Cox regression analysis, covarying for age and KPS, on mean delayed timepoint and aggregate SELFI+ scores as calculated above (e.g., using SPSS v25.0 software available from IBM). P-values less than 0.05 were considered statistically significant.

In the experiment, the early timepoint was defined by SWI within 20 minutes of intravenous ferumoxytol administration. Delayed timepoint was defined by SWI obtained approximately 24 hours following intravenous ferumoxytol administration. Exclusion criteria included the absence of delayed timepoint ferumoxytol-enhanced T1-weighted or SWI MRI. The experiment included subjects meeting the following inclusion criteria: (1) histologically confirmed diagnosis of glioblastoma (World Health Organization classification, grade IV glioma), (2) documentation of IDH-1 mutational and MGMT promoter methylation status (R132H; Clinical Laboratory Improvement Amendments (CLIA) #38D2018256), (3) a Karnofsky Performance Score (KPS)>50, (4) prior CRT utilizing Stupp protocol, (5) GBCA-MRI demonstrating evidence of disease progression according to RANO criteria, (6) GBCA-MRI within 72 hours prior to ferumoxytol-MRI, and (7) SWI performed prior to and following intravenous ferumoxytol administration.

Prior to disease progression, patients underwent the same current standard of care (maximal safe resection followed by CRT only). Ferumoxytol-enhanced MRI was performed at time of disease progression in all patients. Review of the medical record established either glioblastoma recurrence or pseudoprogression as the etiology for disease progression.

Twenty-three patients with radiographic progression (16 disease recurrence and 7 with inflammatory pseudoprogression) were comprised of 19 males (83%) with a mean age of 55 years (range, 23 to 76 years). Positive SELFI values specifically correlated with he delayed timepoint map values (r₍₂₁₎=0.87, p<0.001), suggesting an association with accumulated extravascular ferumoxytol. Conversely, negative SELFI values significantly correlated with both the early (r₍₂₁₎=−0.79, p<0.01) and delayed timepoint map values (r₍₂₁₎=−0.48, p=0.02), demonstrating that intravascular ferumoxytol signal persists in the delayed timepoint. Tumor-wide SELFI+ metric demonstrated a trend toward significance (B=2.98, Wald=2.57, p=0.1). In the SELFI+ multivariate model, higher KPS and age were associated with longer and shorter survival (p=0.02 and 0.05, respectively).

Sixteen patients (70% of the cohort) were diagnosed with recurrent tumor. Four of these patients underwent standard of care re-resection to establish disease status via histological analysis. The remaining 19 patients were diagnosed with either pseudoprogression (5 IDH wild type, 2 IDH mutated) or recurrent tumor via integration of patient clinical course and serial GBCA-MRI. Thirteen patients received dexamethasone (mean±standard deviation, 3.74±4.56 mg) concurrent with ferumoxytol administration. Four patients with tumor recurrence received bevacizumab (10 mg/kg) prior to or the same day as ferumoxytol administration. Dexamethasone and bevacizumab were administered to reduce the clinical side effects of vasogenic edema.

The cohort-wide root volumes of GBCA-enhancement significantly exceeded those of delayed ferumoxytol enhancement (paired t₍₂₂₎=2.25, p=0.03,), as did the mean normalized T1 image intensity within enhancing ROIs (paired t₍₂₂₎=2.77, p=0.01). Despite significant differences in enhancement volumes and intensities, GBCA and ferumoxytol T1 enhancement volumes were significantly correlated (r₍₂₁₎=0.87, p<0.01) as were normalized image intensities (r₍₂₁₎=0.87, p=0.01). Finally, the enhancing volumes in GBCA and ferumoxytol T1 were correlated with their respective normalized intensities (r₍₂₁₎=0.51 and 0.53, p<0.01, respectively). All findings presented here were similar in both GBCA- and ferumoxytol-enhancing ROIs; therefore, only values derived from ferumoxytol-enhancing ROIs are reported, unless GBCA and ferumoxytol are directly compared.

Early and delayed timepoint SWI measurements were significantly correlated, suggesting that intravascular ferumoxytol signal contaminates delayed measurements when using the standard uncorrected approach (r₍₂₁₎=0.56, p<0.01). An example of early timepoint, delayed timepoint, SELFI maps and corresponding voxel-wise histograms from a single patient with disease recurrence can be found in FIG. 2.

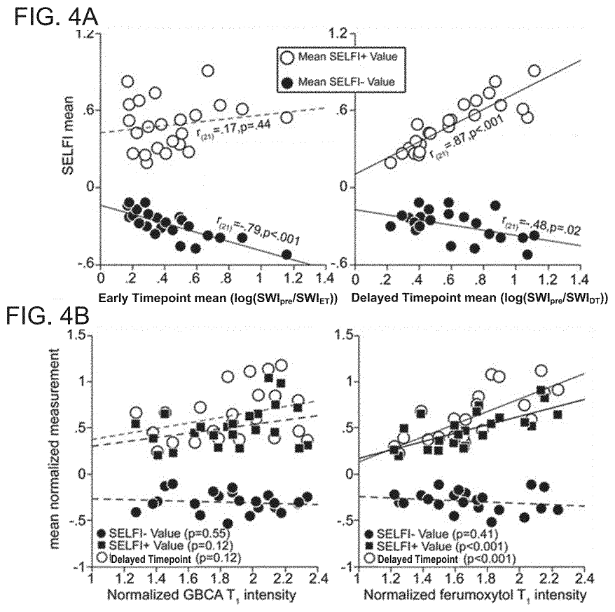

FIGS. 4A and 4B show graphs that reflect mean SELFI+ and SELFI− values. Both SELFI+ (extravascular) and SELFI− (remaining intravascular signal at the delayed imaging timepoint) were correlated with delayed timepoint SWI measurements (r₍₂₁₎=0.87 and −0.48, respectively, p<0.03, FIG. 4A right panel graph), confirming the contamination of intravascular ferumoxytol in delayed timepoint SWI measurements; however, only SELFI− values were significantly correlated with early timepoint SWI measurements confirming localization of intravascular signal (r₍₂₁₎=0.87, p<0.001, FIG. 4A left panel graph).

In FIG. 4A, intravascular ferumoxytol signal (SELFI−, solid circle) is observed in both the early timepoint (left panel graph) and the delayed timepoint map (right panel graph), contaminating the signal on the latter. Conversely, SELFI+ values (empty circles) are only correlated with delayed timepoint map values (right panel graph) suggesting the metric is not present in the early timepoint of imaging.

FIG. 4B shows a relationship of GBCA (left panel graph) and delayed ferumoxytol (right panel graph) T1 intensity to SELFI metrics. SELFI+ values (solid squares) and delayed timepoint map mean values (empty circles) demonstrates a significant correlation only with delayed ferumoxytol (right panel graph) and not GBCA T1 enhancing signal (left panel graph) intensity. SELFI− values (solid circles) are not correlated with enhancement signal intensity. Note that statistically significant (p<0.05) regressions are shown as solid lines and non-significant regressions are represented by dashed lines. Each point on the graph represents a patient's mean imaging value.

Normalized signal intensity of delayed timepoint ferumoxytol T1 enhancement was correlated with SELFI+(extravascular) measurements and delayed timepoint SWI maps (r₍₂₁₎=0.68 and 0.75, p<0.001, respectively), but not with SELFI− (intravascular) measurements (r₍₂₁₎=−0.19, p>0.3; FIG. 4B, right panel graph). Normalized signal intensity of GBCA T1 enhancement was not correlated with delayed timepoint SWI maps or SELFI measures (r₍₂₁₎=0.33, 0.33, and −0.13, p>0.1; FIG. 4B, bottom left) suggesting that the functional significance of the magnitude of delayed ferumoxytol T1 enhancement differs from GBCA T1 enhancement.

The overall cohort survival ranged from 5.83 months to 91.0 months (median=16.6 months). Cox regression survival analysis of the 19 patients with IDH wild-type disease progression (5 pseudoprogression, 14 disease recurrence) was non-significant for the delayed timepoint metric (B=−0.13, Wald=0.017, p>0.8), though it approached significance for the tumor-wide SELFI+ metric (B=2.98, Wald=2.57, p=0.1). Higher KPS and age were significantly associated with longer and shorter survival, respectively, in the SELFI+ metric multivariate model (p=0.02 and 0.05, respectively) but not in the delayed timepoint model (p=0.06 and 0.06) further demonstrating the value of isolating inflammation-specific signal and the SELFI metric.

SELFI technique localizes extravascular ferumoxytol accumulation at the 24-hour delayed imaging timepoint by reducing contaminating intravascular signal. SELFI+ metric determines glioblastoma outcomes by serving as a more specific metric of tumor macrophage content when compared to the current use of uncorrected T1- or susceptibility-weighted techniques.

SELFI maps facilitate distinguishing extravascular from intravascular ferumoxytol pools in twenty-three patients with CRT-treated glioblastoma at the time of clinical disease progression. The results suggest that SELFI+ values represent accumulated extravascular ferumoxytol after 24 hours, and that SELFI− values delineate the persistence of delayed intravascular ferumoxytol. Higher aggregate SELFI+ values were found to trend towards lower overall survival within this cohort. These observations suggest that both T1 and T2* MRI signal change in the delayed timepoint is partially a reflection of persistent contamination by intravascular ferumoxytol and precludes the accurate assessment of the accumulation of extravascular contrast. SELFI maps provide an approach to localize extravascular ferumoxytol contrast accumulation in the delayed timepoint by eliminating intrinsic tissue and intravascular signal, which suggests that this technique may serve as a more specific imaging metric for localizing TAMs within the glioblastoma immune microenvironment when compared to the current use of uncorrected T1- or susceptibility-weighted imaging subtraction techniques. The SELFI technique presented here advances neuro-oncological research and practice by providing a clinically feasible biomarker of accumulating phagocytic cells of the CNS including TAMs and provides a metric of survival in patients with Stupp protocol treated IDH wild type glioblastoma.

Prior investigations have explored the localization of TAMs using T2*-weighted imaging based on the inverse linear relationship with R2* signal relaxation rates, demonstrating that macrophage infiltration and microglia activation within injured central nervous system tissue are responsible for the accumulation of iron oxide nanoparticles. However, previous reports have not accounted for intrinsic tissue (iron as a byproduct of prior tissue bleeding) or persistent intravascular ferumoxytol signal. If ferumoxytol-associated MRI signal changes are dependent upon transport across the blood brain barrier in the context of activated innate immunity, accurate quantification of brain parenchymal TAMs requires an account of residual intravascular signal. SELFI is one method for deconvolving intravascular and extravascular signal contributions observed within delayed timepoint ferumoxytol-enhanced MRI.

The techniques of this disclosure are valuable in future in vivo investigations of the immune microenvironment within glioblastoma. Glioblastoma cells have complex inhibitory mechanisms to suppress and escape local immune surveillance. Glioblastoma secretes immune suppressive cytokines (PGE2, IL-10, TGF-β) and directly modulates innate immune cells (microglia and TAMs) to inhibit the expansion of effector T cells. The consequence of immune suppression is the establishment of a pro-tumoral immunosuppressive microenvironment that promotes unchecked glioblastoma growth. Functionally, the immunosuppressive microenvironment serves a biological feature that contributes to the development of glioblastoma therapeutic resistance. The noninvasive quantification of the tumor immune microenvironment could provide additional diagnostic and prognostic information beneficial to the planning and monitoring of therapy, and SELFI is one molecular imaging approach by which the glioblastoma innate immune microenvironment could be characterized. The SELFI methodology facilitates TAM delineation within the glioblastoma immune microenvironment and improve clinical management by expediting definitive treatment of glioblastoma patients based upon the degree of immune infiltrate. Techniques of this disclosure are also applicable to investigations of diseases other than glioblastoma and other cancers.

The aforementioned experimentation shows that SELFI+ values are prognostic of overall survival in patients with CRT treated IDH wild type glioblastoma. The development of a clinically prognostic biomarker of the tumor immune environment is a critical gap in knowledge in the era of immunotherapy. Novel therapeutic techniques aimed at immune augmentation require an appropriate molecular imaging metric to demonstrate changes in the targeted biological mechanism. Immune checkpoint blockade leads to an increased anti-tumor immune response, which results in therapy-mediated inflammation. The interaction between tumor and cytotoxic T-cells is considered the key target for checkpoint inhibition, but emerging evidence suggests checkpoint blockade also influences therapeutic efficacy through innate immunity. Preliminary reports suggest that checkpoint efficacy is characterized by marked pro-inflammatory lymphohistiocytic infiltration. Given the potential importance of macrophage activation in glioblastoma therapy, the development of a biologically specific imaging metric of pro-tumoral and pro-inflammatory macrophage accumulation is desirable. The utility of delayed ferumoxytol MRI in subjects receiving standard of care CRT with or without concurrent check point inhibitors in primary and metastatic brain tumors (NCT00103038, NCT03347617 and NCT03325166) has been analyzed. This approach provides additional prognostic and predictive value.

Finally, recent findings describe strong correlations between the volume and intensity of GBCA and delayed timepoint ferumoxytol T1 enhancement. T1 enhancement characteristics of ferumoxytol are dependent upon multiple factors, including but not limited to BO field strength, dosage, and imaging time from administration. The similarity between the GBCA and ferumoxytol enhancement supports the use of ferumoxytol as an alternate MRI contrast agent for the diagnosis of CNS pathologies when standard of care GBCA administration is precluded, such as acute kidney injury, while the differences between the extent and intensity of enhancement between the two agents is indicative of differential mechanisms of uptake and accumulation.

Embodiments of this disclosure describe, for example, a newly developed SELFI technique for the differentiation of extravascular ferumoxytol contrast signal from residual intravascular signal at the 24-hour delayed timepoint imaging time point within CRT treated glioblastoma at the time of disease progression. SELFI improves upon current techniques by eliminating contributions from intrinsic tissue and intravascular signal through voxel-wise subtraction of early- and delayed-timepoint ferumoxytol maps. This methodology provides a spatially specific biomarker for the accumulation of phagocytic cells such as TAMs within the glioblastoma immune microenvironment and has the potential to inform treatment at each step of the clinical management of glioblastoma.

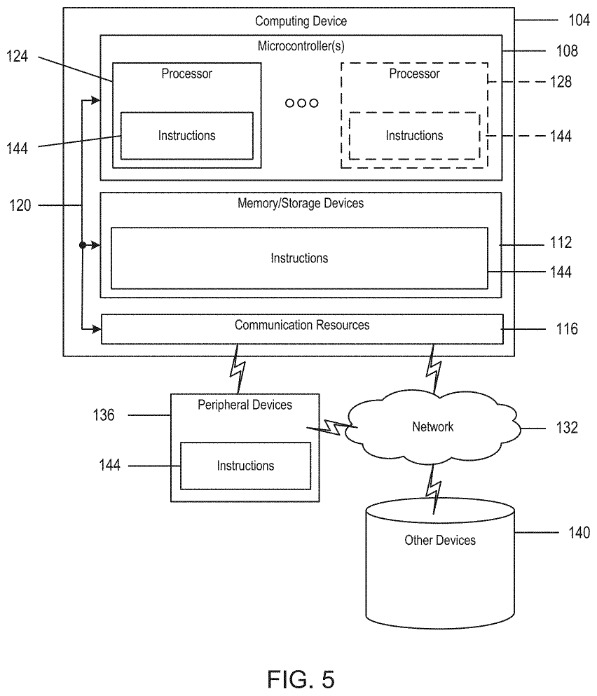

Embodiments described herein may be implemented in any suitably configured hardware and software resources of computing device 104, as shown in FIG. 5. And various aspects of certain embodiments are implemented using hardware, software, firmware, or a combination thereof, for reading instructions from a machine- or computer-readable non-transitory storage medium and thereby performing one or more of the methods realizing the disclosed algorithms and techniques. Specifically, computing device 104 can include one or more microcontrollers 108, one or more memory/storage devices 112, and one or more communication resources 116, all of which are communicatively coupled via a bus 120 or other circuitry.

For example, in some embodiments, computing device 104 detects an immune factor (e.g., corresponding to microphages, corresponding tomicroglia, or other immune factor(s)) responsive to a contrast agent. The immune factor may be responsive to treatment of a disease, where the treatment may be chemoradiotherapy and the disease may be cancer. For example, the cancer may be glioblastoma of the brain.

In some embodiments, computing device 104 receives one or more of pre-contrast image data, early timepoint image data, and delayed timepoint image data. In some embodiments, one or more of the pre-contrast image data, early timepoint image data, and delayed timepoint image data are susceptibility weighted image (SWI) sequence data. In some embodiments, one or more of the pre-contrast image data, early timepoint image data, and delayed timepoint image data are MRI image sequence data or any other type of image data that is sensitive to the contrast agent.

In some embodiments, the receiving occurs via a network 132. For example, image data may be received from another device 140 via network 132. Alternatively, in other embodiments the receiving occurs directly, without use of network 132, from a peripheral device 136. The direct reception may occur via wired communication (e.g., for communication via a Universal Serial Bus (USB)), Near Field Communication (NFC), Bluetooth® (e.g., Bluetooth® Low Energy), or other forms of wireless communication, for example. In some embodiments, the image data is received from other device 140 via both network 132 and peripheral device 136. In some embodiments, the image data is received from peripheral device 136 via network 132. In some embodiments, two of the pre-contrast image data, early timepoint image data, and delayed timepoint image data are received by computing device 104 via network 132 while the other image data not received via network 132 is received by computing device 104 directly via peripheral device 136. In some embodiments, two of the pre-contrast image data, early timepoint image data, and delayed timepoint image data are received by computing device 104 via direct communication from peripheral device 136 while the other image data not received via direct communication is received by computing device 104 via network 132.

In some embodiments, the pre-contrast image data represents image data at an initial time related to intravenous administration of the contrast agent, the early timepoint image data represents image data at an early timepoint following intravenous administration of the contrast agent, and the delayed timepoint image data represents image data at delayed timepoint following intravenous administration of the contrast agent. In some embodiments, the image data is generated by MRI imaging. Image data generated by any other imaging techniques are also contemplated by embodiments of the disclosure. In some embodiments, image data is extracted from video data as frames that form the video data.

In some embodiments, computing device 104 generates an early timepoint map using a comparison of the pre-contrast image data to the early timepoint image data. The early timepoint map represents intravascular contrast. The intravascular contrast is contrast situated inside blood vessels in tissue. In some embodiments, computing device 104 generates a delayed timepoint map using a comparison of the pre-contrast image data to the delayed timepoint image data. The delayed timepoint map represents the intravascular contrast and extravascular contrast, the extravascular contrast indicating the immune factor. The extravascular contrast is contrast situated outside blood vessels in tissue.

In some embodiments, computing device 104 generates a combined immune factor map by voxel-wise subtraction of the early timepoint map from the delayed timepoint map. Here, the combined immune factor map suppresses the representation of the intravascular contrast of the delayed timepoint map and localizes the representation of the extravascular contrast of the delayed timepoint map indicating the immune factor.

In some embodiments, microcontroller(s) 108, includes, for example, one or more processors 124 (shared, dedicated, or group), one or more optional processors (or additional processor core) 128, one or more ASIC or other controller to execute one or more software or firmware programs, one or more combinational logic circuit, or other suitable components that provide the described functionality.

In some embodiments, memory/storage devices 112 includes main memory, cache, flash storage, or any suitable combination thereof. A memory device 112 may also include any combination of various levels of non-transitory machine-readable memory including, but not limited to, electrically erasable programmable read-only memory (EEPROM) having embedded software instructions (e.g., firmware), dynamic random-access memory (e.g., DRAM), cache, buffers, or other memory devices. In some embodiments, memory is shared among the various processors or dedicated to particular processors.

In some embodiments, communication resources 116 include physical and network interface components or other suitable devices to communicate with one or more peripheral devices 136. In one example, communication resources 116 communicates via a network 132 with one or more peripheral devices 136 (e.g., computing devices, imaging devices, etc.) or one or more other devices 140 (e.g., other computing devices, other imaging devices). In some embodiments, network 132 uses one or more of a wired communication (e.g., for communication via a Universal Serial Bus (USB)), cellular communication, Near Field Communication (NFC), Bluetooth® (e.g., Bluetooth® Low Energy), Wi-Fi®, and any other type of wired or wireless communication. In some embodiments, communication resources 116 includes one or more of wired communication components (e.g., for coupling via a Universal Serial Bus (USB)), cellular communication components, Near Field Communication (NFC) components, Bluetooth® components (e.g., Bluetooth® Low Energy), Wi-Fi® components, and components for any other type of wired or wireless communication.

In some embodiments, instructions 144 comprises software, a program, an application, an applet, an app, or other executable code for causing at least any of microcontroller(s) 108 to perform any one or more of the methods discussed herein. For example, instructions 144 can facilitate receiving (e.g., via communication resources 116) image data discussed previously. Instructions 144 can then facilitate the processing described in accordance with the embodiments of this disclosure.

In some embodiments, instructions 144 reside completely or partially within one (or more) of microcontroller(s) 108 (e.g., within a processor's cache memory), memory/storage devices 112, or any suitable combination thereof. Furthermore, any portion of instructions 144 may be transferred to computing device 104 from any combination of peripheral devices 136 or the other devices 140. Accordingly, memory of microcontroller(s) 108, memory/storage devices 112, peripheral devices 136, and the other devices 140 are examples of computer-readable and machine-readable media.

In some embodiments, instructions 144 also, for instance, comprise one or more physical or logical blocks of computer instructions, which may be organized as a routine, program, object, component, data structure, text file, or other instruction set facilitating one or more tasks or implementing particular data structures or software modules. A software module, component, or library may include any type of computer instruction or computer-executable code located within or on a non-transitory computer-readable storage medium. In certain embodiments, a particular software module, component, or programmable rule comprises disparate instructions stored in different locations of a computer-readable storage medium, which together implement the described functionality. Indeed, a software module, component, or programmable rule may comprise a single instruction or many instructions, and may be distributed over several different code segments, among different programs, and across several computer-readable storage media. Some embodiments can be practiced in a distributed computing environment where tasks are performed by a remote processing device linked through a communications network.

In some embodiments, instructions 144, for example, include .Net and C libraries providing machine-readable instructions that, when executed by processor 124, cause processor 124 to perform image analysis techniques in accordance with the present disclosure, including detecting an immune factor response to a contrast agent.

In some embodiments, image data used by image analysis techniques of the present disclosure are received by computing device 104 from one or more other devices 140, one or more peripheral devices 136, or a combination of one or more other devices 140 and peripheral devices 136. In some embodiments, one or both of other devices 140 and peripheral devices 136 are MRI imaging devices or any other kind of imaging or video device that capture the image data, video data, or both image data and video data. In some embodiments, one or both of other devices 140 and peripheral devices 136 are computing devices that store the image, video data, or both image data and video data.

In some embodiments, computing device 104 is an MRI device or any other kind of imaging or video device that captures image data, video data, or both image data and video data used by the image analysis techniques of the present disclosure. Thus, in some embodiments, computing device 104 itself receives image data and performs image analysis techniques of the present disclosure using that received image data.

In some embodiments, the images used by the image analysis techniques of the present disclosure are image frames extracted from video data.

Skilled persons will now appreciate that many changes may be made to the details of the above-described embodiments without departing from the underlying principles of the invention. The scope of the present invention should, therefore, be determined by the following claims.

1. A method for detecting an immune factor responsive to a contrast agent comprising:
receiving pre-contrast image data, early timepoint image data, and delayed timepoint image data, the pre-contrast image data representing image data at an initial time related to intravenous administration of the contrast agent, the early timepoint image data representing image data at an early timepoint following intravenous administration of the contrast agent, and the delayed timepoint image data representing image data at delayed timepoint following intravenous administration of the contrast agent;
generating an early timepoint map using a comparison of the pre-contrast image data to the early timepoint image data, and generating a delayed timepoint map using a comparison of the pre-contrast image data to the delayed timepoint image data, the early timepoint map representing intravascular contrast, and the delayed timepoint map representing the intravascular contrast and extravascular contrast, the extravascular contrast indicating the immune factor; and
generating a combined immune factor map by voxel-wise subtraction of the early timepoint map from the delayed timepoint map, the combined immune factor map suppressing a representation of the intravascular contrast of the delayed timepoint map and localizing a representation of the extravascular contrast of the delayed timepoint map indicating the immune factor.

2. The method of claim 1, wherein the receiving comprises receiving, via a network, one or more of the pre-contrast image data, early timepoint image data, and delayed timepoint image data.

3. The method of claim 1, wherein the receiving comprises receiving, via direct communication, one or more of the pre-contrast image data, early timepoint image data, and delayed timepoint image data.

4. The method of claim 1, wherein the receiving comprises receiving, from an imaging device, one or more of the pre-contrast image data, early timepoint image data, and delayed timepoint image data.

5. The method of claim 4, wherein the imaging device is an MRI imaging device.

6. The method of claim 1, wherein the receiving comprises receiving, from a computing device, one or more of the pre-contrast image data, early timepoint image data, and delayed timepoint image data.

7. The method of claim 1, wherein the initial time related to the intravenous administration of the contrast agent is prior to the intravenous administration of the contrast agent.

8. The method of claim 1, wherein the initial time related to the intravenous administration of the contrast agent is about zero seconds to about 15 minutes after the intravenous administration of the contrast agent.

9. The method of claim 1, wherein the early timepoint is about 15 minutes to about 20 minutes following the intravenous administration of the contrast agent.

10. The method of claim 1, wherein the delayed timepoint is about 24 hours to about 72 hours following the intravenous administration of the contrast agent.

11. The method of claim 1, wherein the intravascular contrast is contrast situated inside blood vessels in tissue.

12. The method of claim 1, wherein the extravascular contrast is contrast situated outside blood vessels.

13. The method of claim 1, wherein the contrast agent includes metal nanoparticles.

14. The method of claim 13, wherein the metal nanoparticles are iron nanoparticles.

15. The method of claim 13, wherein the metal nanoparticles are gold nanoparticles.

16. The method of claim 1, wherein the immune factor corresponds to microphages.

17. The method of claim 1, wherein the immune factor corresponds to microglia.

18. The method of claim 1, further comprising applying an enhancement mask to a region of interest in the early timepoint map and the delayed timepoint map.

19. The method of claim 18, further comprising generating a positive immune factor map by calculating a sum of voxels encompassed by the enhancement mask in the combined immune factor map that are greater than zero.

20. The method of claim 19, further comprising scaling the calculated sum of voxels.

21. The method of claim 20, wherein the scaling normalizes the calculated sum of voxels.

22. The method of claim 1, wherein the immune factor is responsive to treatment of a disease.

23. The method of claim 22, wherein the treatment is chemoradiotherapy.

24. The method of claim 22, wherein the disease is cancer.

25. The method of claim 22, wherein the treatment is chemoradiotherapy and the disease is glioblastoma.

26. The method of claim 1, wherein each of the pre-contrast image data, early timepoint image data, and delayed timepoint image data is susceptibility-weighted image sequence data.

27. The method of claim 1, wherein each of the pre-contrast image data, early timepoint image data, and delayed timepoint image data is MRI image sequence data or any other type of image data that is sensitive to the contrast agent.
